(19) United States
(12) Patent Application Publication
(10) Pub. No.: US 2008/0050334 A1
(43) Pub. Date: Feb. 28, 2008

(54) Cosmetic composition comprising an extract of emblica officinalis, a hydroxy acid, and an N-substituted aminosulfonic acid, and methods of using same

(75) Inventors: Isabelle Hansenne, Westfield, NJ (US); Hani Fares, Somerset, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 11/510,364

(22) Filed: Aug. 24, 2006

(51) Int. Cl. A61K 8/97

(52) U.S. Cl. 424/74; 424/769

(57) ABSTRACT

A cosmetic composition useful for the depigmentation of skin containing an extract of Emblica officinalis, at least one hydroxy acid, and at least one N-substituted aminosulfonic acid having the structural formula (I):

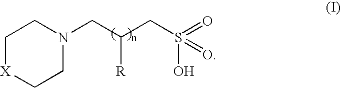

### BACKGROUND OF THE INVENTION

Hyperpigmentation in skin is generally the result of increased melanin deposition in epidermal cells. Hyperpigmentation of skin is associated with freckles, senile lentigo, lentigines, melasma, post-inflammatory hyperpigmentation, sunburn, phototoxic reactions and other conditions.

Melanin production is mediated by a tyrosinase enzyme. In order to prevent melanin production, tyrosinase inhibitors need be used. Several tyrosinase inhibitors are available on the marketplace, including hydroquinone, kojic acid and arbutin. However, there are reports of disadvantages associated with each of these products. For example, the bioavailability of kojic acid and arbutin is not very pronounced and thus kojic acid and arbutin are of marginal efficacy. Hydroquinone is reported to be oxidized by air, light and tyrosinase itself. Depigmenting compositions with hydroquinone also tend to cause irritation. Extracts of Emblica officinalis have been identified as being effective depigmentation agents. In that respect, FR2730408 discloses and claims skin lightening compositions containing extracts of Emblica officinalis and α-hydroxy acids. Alpha-hydroxy acids, however, tend to be harsh on the skin at higher concentrations.

Therefore, it is an object of the present invention to provide a skin depigmentation composition which is effective, yet mild, and without the above limitations.

### BRIEF SUMMARY OF THE INVENTION

A first aspect of the present invention is directed to a skin depigmenting composition comprising:

(a) an extract of Emblica officinalis;
(b) at least one hydroxy acid;
(c) at least one N-substituted aminosulfonic acid; and
(d) optionally, at least one photoprotective agent.

A second aspect of the present invention is directed to a method of depigmenting skin, comprising applying the above disclosed composition to the skin.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

Emblica officinalis

As used herein, the terms “an extract of Emblica officinalis,” “emblica extract” or “an extract of emblica” mean any extract of the fruit of the Emblica officinalis or Phyllanthus emblica tree. Such extracts are described, for example, on pp. 175-176 of the Handbook of Ayurvedic Medicinal Plants, L. D. Kapoor, CRC Press, Inc., 1990. Additionally, such extracts are described in U.S. Pat. No. 6,261,605 B1 to Singh-Verma, the entire contents of which are hereby incorporated by reference. Such extracts may be prepared by any method known to those skilled in the art. In addition, the terms “an extract of Emblica officinalis,” “emblica extract” and “an extract of emblica” mean any compounds or mixtures of compounds that are isolated or purified from crude extracts of the fruit of the tree. Such compounds or mixtures of compounds may be isolated or purified by any method known to those skilled in the art. For example, compositions comprising emblica extract, as well as its individual chemical components are described in U.S. Pat. No. 6,124,268, the entire contents of which are hereby incorporated by reference.

Emblica officinalis is generally present in the cosmetic composition in an amount ranging from 0.05% to 10% by weight, preferably from 0.1% to 5% by weight and more preferably from 0.1% to 3% by weight, based on the weight of the composition.

Hydroxy Acid

Hydroxy acids are acids which have at least one hydroxy group in their molecules. Hydroxy acids may be characterized as α-hydroxy acids, β-hydroxy acids or poly-hydroxyacids.

As used herein, the term “α-hydroxy acids” means organic compounds that contain a hydroxy group in a position alpha to the carboxyl group. Such acids can be either naturally derived from various fruits, or they may be synthetic in nature.

Examples of suitable α-hydroxy acids that may be used according to the present invention include, but are not limited to, glycolic acid, lactic acid, tartaric acid, citric acid, malic acid, and mandelic acid.

As used herein, the term “β-hydroxy acids” means organic acids that contain a hydroxy group in a position beta to the carboxyl group.

Examples of suitable β-hydroxy acids that may be used according to the present invention include, but are not limited to, salicylic acid, salicylic acid derivatives which may include, but are not limited, to 5-n-octanoylsalicylic acid and 5-n-dodecanoylsalicylic acid such as described in U.S. Pat. No. 4,767,750, and U.S. Pat. No. 5,558,871 the entire contents of which are hereby incorporated by reference. Other β-hydroxy acids which may be used include other derivatives of salicylic acid such as sodium salicylate, or willow extract, beta-hydroxybutanoic acid, tropic acid, and trethocanic acid.

As used herein, the term “poly-hydroxy acids” means organic acids that contain more than one hydroxyl group and a carboxyl group.

Examples of suitable poly-hydroxy acids that may be used according to the present invention include, but are not limited to, glyceric, dihydroxybutyric, ascorbic, glucuronic, mannuronic, tartronic, hydroxymalonic, malic, citramalic, hydroxyglutaric, tartaric, hydroxyfumaric, hydroxymaleic, dihydroxy maleic, dihydroxy fumaric, dihydroxy tartaric, citric and isocitric acids.

The hydroxy acid is generally present in the cosmetic composition in an amount ranging from 0.1% to 30% by weight, preferably from 0.2% to 20% by weight and more preferably from 0.25% to 15% by weight, based on the weight of the composition.

N-Substituted Aminosulfonic Acid

The N-substituted aminosulfonic acids suitable for use in the present invention are those having the structural formula (I):

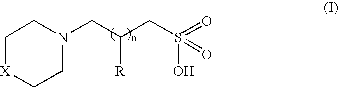

in which R is a hydrogen atom, —OH or —NH₂; X is an oxygen atom, a group corresponding to:

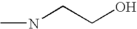

or a group corresponding to:

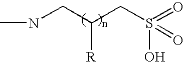

and n is an integer from 0 to 3.

Preferably, the N-substituted aminosulfonic acid compounds of formula (I) according to the invention are those in which:

R is a hydrogen atom, —OH or —NH₂;

X is an oxygen atom, a group corresponding to:

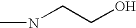

or a group corresponding to:

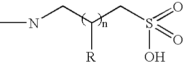

and n is equal to 0 or 1.

More preferably, derivatives of formula (I) may include, but are not limited to:

4-(2-hydroxyethyl)piperazine-1-ethanesulfonic acid which corresponds to the following formula:

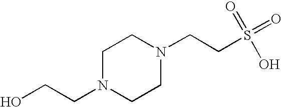

4-(2-hydroxyethyl)piperazine-1-(2-hydroxypropane-sulfonic acid) which corresponds to the following formula:

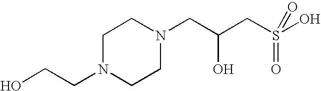

4-(2-hydroxyethyl)piperazine-1-propanesulfonic acid which corresponds to the following formula:

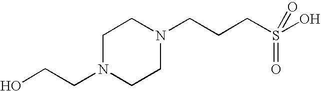

3-morpholinopropanesulfonic acid which corresponds to the following formula:

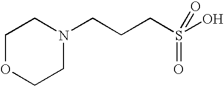

2-morpholinoethanesulfonic acid which corresponds to the following formula:

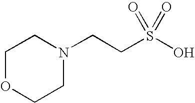

piperazine-1,4-bis(2-ethanesulfonic acid) which corresponds to the following formula:

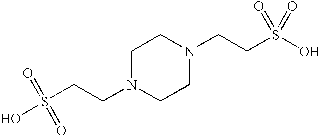

piperazine-1,4-bis(2-hydroxypropanesulfonic acid) which corresponds to the following formula:

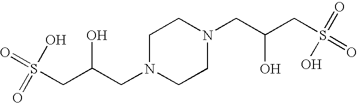

The most preferred N-substituted aminosulfonic acids are piperazine-1,4-bis(2-hydroxypropanesulfonic acid), piperazine-1,4-bis(2-ethanesulfonic acid), and 4-(2-hydroxyethyl)piperazine-1-ethanesulfonic acid, the latter also being known under the acronym HEPES.

The N-substituted aminosulfonic acid is generally present in the cosmetic composition in an amount ranging from 0.1% to 20% by weight, preferably from 0.1% to 10% by weight and more preferably from 0.1% to 5% by weight, based on the weight of the composition.

The compositions may be provided in all dosage forms conventionally used for topical application and, in particular, in the form of: (i) dispersions of the lotion or gel type, (ii) emulsions with a liquid or semi-liquid consistency of the milk type, obtained by dispersion of a fatty phase in an aqueous phase (O/W) or vice versa (W/O), (iii) suspensions or emulsions with a soft, semi-solid or solid consistency of the cream or gel type, (iv) multiple emulsions (W/O/W or O/W/O), (v) microemulsions, (vi) vesicular dispersions of ionic and/or nonionic type, or (vii) wax/aqueous phase dispersions. These compositions are prepared according to methods known to those of ordinary skill in the art of cosmetics or dermatological formulations.

The composition of the present invention, in the form of an emulsion, contains oils which include, but are not limited to silicone oils, which may be volatile or nonvolatile, hydrocarbon oils, which may be volatile or nonvolatile, or vegetable oils. These emulsions can additionally comprise non-oily fatty substances, such as shea butter, silicone gums, esters of fatty acids and of fatty alcohols, fatty acids and fatty alcohols.

The composition of the present invention, in the form of an emulsion, can comprise at least one compound useful as an emulsifier. Suitable emulsifiers that can be used according to the present invention include, but are not limited to, nonionic, cationic, anionic, and zwitterionic emulsifiers. Suitable emulsifiers according to the present invention include, but are not limited to, acyl lactylates, alkyl phosphates, carboxylic acid copolymers, esters and ethers of glucose, esters of glycerin, esters of propylene glycol, esters of sorbitan anhydrides, esters of sorbitol, ethoxylated ethers, ethoxylated alcohols, fatty acid amides, fatty acid esters of polyethylene glycol, fatty esters of polypropylene glycol, polyoxyethylene fatty ether phosphates, soaps, fatty alcohols, and mixtures thereof. Other emulsifiers that may be used include, but are not limited to, PPG-2 isoceteth-20 acetate, ceteareth-20, ceteth-10, cetyl phosphate, diethanolamine cetyl phosphate, glyceryl stearate, PEG-100 stearate, polyethylene glycol 20 sorbitan monolaurate, polyethylene glycol 5 soya sterol, polysorbate 60, polysorbate 80, potassium cetyl phosphate, PPG-2 methyl glucose ether distearate, steareth-20, and mixtures thereof. In one embodiment, the emulsifier is chosen from cetearyl alcohol, PPG-2 myristyl ether propionate, PPG-15 stearyl ether, glyceryl stearate, PEG-100 stearate and mixtures thereof. For examples of other suitable emulsifiers that can be used according to the present invention, see, for example, McCutcheon's, Detergents and Emulsifiers, North American Edition (2003), Allured Publishing Corporation; and U.S. Pat. Nos. 5,011,681, 4,421,769, and 3,755,560, the entire contents of which are hereby incorporated by reference.

The compositions according to the invention can also comprise at least one emollient. Suitable emollients include, but are not limited to, branched hydrocarbons, carboxylic acid and alcohol esters, volatile and non-volatile silicone oils, and mixtures thereof. See, for example, U.S. Pat. No. 4,919,934, the entire contents of which are hereby incorporated by reference. Examples of emollients that can be used according to the present invention include, but are not limited to, at least one of isostearyl neopentanoate, isopropyl palmitate, ethylhexyl palmitate and propylene glycol isoceteth-3 acetate.

It is highly recommended that a suitable photoprotective agent be part of the composition of the present invention.

Suitable organic photoprotective agents include, but are not limited to, anthranilates; cinnamic derivatives; dibenzoylmethane derivatives; salicylic derivatives; camphor derivatives; triazine derivatives; benzophenone derivatives; β,β-diphenylacrylate derivatives; benzotriazole derivatives; benzalmalonate derivatives; benzimidazole derivatives; imidazolines; bisbenzoazolyl derivatives; p-aminobenzoic acid (PABA) derivatives; methylenebis(hydroxyphenylbenzotriazole) derivatives; benzoxazole derivatives; screening polymers and screening silicones, such as those disclosed in particular in patent application WO 93/04665, the entire contents of which are incorporated herein by reference; dimers derived from α-alkylstyrene; 4,4-diarylbutadienes, and mixtures thereof.

The organic photoprotective agents more particularly preferred may be chosen from the following compounds (CTFA names or chemical names):
Ethylhexyl Salicylate,
Ethylhexyl Methoxycinnamate,
Octocrylene,
Phenylbenzimidazole Sulfonic Acid,
Benzophenone-3, -Benzophenone-4,
Benzophenone-5,
4-Methylbenzylidene Camphor,
Terephthalylidene Dicamphor Sulfonic Acid, also known as Mexoryl SX,
Disodium Phenyl Dibenzimidazole Tetrasulfonate,
2,4,6-Tris(diisobutyl 4′-aminobenzalmalonate)-s-triazine,
Anisotriazine,
Ethylhexyl Triazone,
Diethylhexyl Butamido Triazone,
Methylene Bis-Benzotriazolyl Tetramethyl-butylphenol,
Drometrizole Trisiloxane, also known as Mexoryl XL,
Polysilicone-15,
1,1-Dicarboxy(2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine, and their mixtures.

Suitable inorganic photoprotective agents include, but are not limited to, pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminum stearate. Such nanopigments formed of metal oxides, which may or may not be coated, are disclosed in particular in Patent Applications EP 518,772 and EP 518,773, the entire contents of which are incorporated herein by reference.

The photoprotective agents may be present in the cosmetic composition in an amount ranging from 0.5%% to 35% by weight, preferably from 0.75% to 30% by weight and more preferably from 1% to 20% by weight, based on the weight of the composition. The composition preferably has an SPF value of at least 15, such as 20 or 30 or greater.

The composition of the present invention may also include other various adjuvants commonly used in the cosmetics and dermatological field, such as fillers, in particular polyacrylamide (Nylon) fibers and/or microbeads, silica, optionally in the form of a colloidal dispersion, and/or organic microspheres which are optionally expanded; preservatives such as parabens and/or copreservatives, such as caprylyl glycol; sequestering agents, such as EDTA salts; colorants; fragrances; botanical extracts; antioxidants such as tocopherol and its derivatives; humectants such as glycerin, propylene glycol or butylene glycol; pH adjusters, such as neutralizing agents and/or buffering agents; ethanol; and thickening and gelling agents, in particular acrylamide homo- and co-polymers, acrylic homo- and co-polymers, acrylamidomethylpropanesulfonic acid (AMPS) homo- and co-polymers, silicates, and xanthan gum.

The present invention will be better understood from the examples which follow, all of which are intended for illustrative purposes only and are not meant to unduly limit the scope of the invention in any way.

### EXAMPLES

Inventive Composition

| Phase | Raw Material | % w/w |
|---|---|---|
| A | Water | 47.4 |
| A1 | Glycerin | 2.5 |
| A1 | Thickeners | 0.7 |
| A2 | Humectants | 6.5 |
| A2 | Preservatives | 0.4 |
| B | Ester and silicone emollients | 8.0 |
| B | Emulsifiers | 12.0 |
| B | Antioxidant | 0.5 |
| C | Glycolic Acid (70% w/w solution in | 5.7 |
| | water) | |
| C | Water | 2.0 |
| C | Ammonium Hydroxide solution (20% NH₃) | 2.0 |
| C | Hydroxyethylpiperazine Ethane | 5.0 |
| | Sulfonic Acid (HEPES) | |
| D | Fruit Extract | 0.3 |
| D | Phyllanthus Emblica Fruit Extract | 2.0 |
| D | Water | 5.0 |
| | Total | 100.0 |

Processing Protocol.

The equipment used for a batch size of 1 kilogram was a one liter Greerco Homogenizer and a Caframo mixer.

Phase C (glycolic phase): The 70% solution of glycolic acid and the water were combined. Then the ammonium hydroxide solution was added while mixing, followed by the addition of the HEPES with mixing until solution was clear. Phase C was set aside for later use.

Phase A2 (preservatives): The preservatives were weighed and combined with the humectants. The mixture was heated to about 60° C. and set aside.

Phase A1 (thickeners): The thickeners were dispersed into the glycerin and mixed until evenly distributed.

Phase A+A1+A2: Weigh out Phase A water was weighed and placed in the main batch beaker. Phase A1 (thickeners) was added to the main batch water while mixing at moderate speed. Mixing was continued for 15 minutes to ensure uniform and complete dispersion. Phase A+A1 was heated to about 60° C. and phase A2 (preservatives) added while heat was applied to about 80° C. with mixing.

Phase B: Phase B ingredients were weighed out and combined while heat was applied to about 80° C. with mixing.

Emulsification: Phases A and B were heated to 80° C. Phase B was added slowly into phase A and mixed with a homogenizer for 15 minutes.

The homogenized mixture was cooled while mixing with a sweeper blade. At 45° C., Phase C was added and mixed until uniform. Cooling was continued until the temperature reached 25°-30° C. At 25°-30° C., Phase D (Emblica Extract pre-dispersed in water with the addition of the fruit extract) was added and the mixture was homogenized for about 2 minutes. The formula was cooled to 25° C. and transferred into appropriate packaging.

Comparative composition (2% Hydroquinone)

| | |
|---|---|
| HYDROQUINONE | 2.00 |
| DIMETHICONE/VINYL DIMETHICONE CROSSPOLYMER | 1.20 |
| NYLON-12 | 5.00 |
| CYCLOPENTASILOXANE | 17.28 |
| HUMECTANTS | 29.00 |
| DIMETHICONE | 3.80 |
| PRUNUS ARMENIACA (APRICOT) KERNEL OIL | 3.00 |
| PEG/PPG-18/18 DIMETHICONE | 2.40 |
| PHENYL TRIMETHICONE | 4.00 |
| SODIUM METABISULFITE | 0.15 |
| WATER | 31.82 |
| PRESERVATIVES | 0.35 |
| TOTAL | 100.00 |

A controlled, single center, randomized, double blind, parallel group trial was run to compare the efficacy and tolerability of the inventive composition and that of a 2% hydroquinone composition. This composition was applied daily for 16 weeks to the back of the hands and to the face of female panelists with irregular or patchy discoloration of the skin. At baseline visit, the study clinician graded the severity of hyperpigmented lesions on the face, and back of hands, of test subjects using a visual analog scale with 1=none and 10=severe. Subjects with melasma were rated using a scale from 0=none to 3=severe. Triplicate Chromameter measurements were taken on the same selected hyperpigmented lesions of the face and hands at each visit. Subjects hands were photographed using a digital camera while subjects' face was photographed using VISIA imaging system. Subjects returned at 6 weeks, 12 weeks and 16 weeks for repeat grading, Chromameter measurements and photography. Results show that the inventive composition was just as effective as the comparative composition in treating hyperpigmented lesions, while at the same time significantly lowering the degree of erythema experienced by test subjects.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A cosmetic composition comprising:
(a) an extract of Emblica officinalis;
(b) at least one hydroxy acid;
(c) at least one N-substituted aminosulfonic acid having the structural formula (I): 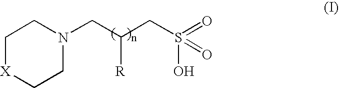
in which R is chosen from a hydrogen atom, —OH or —NH₂; X is chosen from an oxygen atom, a group corresponding to: 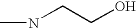
or a group corresponding to: 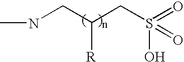
and n is an integer from 0 to 3; and
(d) optionally, at least one photoprotective agent.

2. The composition of claim 1, wherein the extract of Emblica officinalis is present in the composition in an amount of from about 0.05% to about 10% by weight, based on the weight of the composition.

3. The composition of claim 1, wherein the extract of Emblica officinalis is present in the composition in an amount of from about 0.1% to about 3% by weight, based on the weight of the composition.

4. The composition of claim 1, wherein the at least one hydroxy acid is chosen from α-hydroxy acids, β-hydroxy acids, poly-hydroxy acids and their mixtures.

5. The composition of claim 1, wherein the at least one hydroxy acid is glycolic acid.

6. The composition of claim 1, wherein the at least one hydroxy acid is present in the composition in an amount of from about 0.1% to about 30% by weight, based on the weight of the composition.

7. The composition of claim 1, wherein the at least one hydroxy acid is present in the composition in an amount of from about 0.25% to about 15% by weight, based on the weight of the composition.

8. The composition of claim 1, wherein in formula (I) n is equal to 0 or 1.

9. The composition of claim 1, wherein the at least one N-substituted aminosulfonic acid is piperazine-1,4-bis(2-ethanesulfonic acid).

10. The composition of claim 1, wherein the at least one N-substituted aminosulfonic acid is piperazine-1,4-bis(2-hydroxypropanesulfonic acid).

11. The composition of claim 1, wherein the at least one N-substituted aminosulfonic acid is 4-(2-hydroxyethyl)piperazine-1-propanesulfonic acid.

12. The composition of claim 1, wherein the at least one N-substituted aminosulfonic acid is present in the composition in an amount of from about 0.1% to about 20% by weight, based on the weight of the composition.

13. The composition of claim 1, wherein the at least one N-substituted aminosulfonic acid is present in the composition in an amount of from about 0.1% to about 5% by weight, based on the weight of the composition.

14. The composition of claim 1, wherein the photoprotective agent is terephthalylidene dicamphor sulfonic acid.

15. The composition of claim 1, wherein the photoprotective agent is drometrizole trisiloxane.

16. The composition of claim 1, wherein the photoprotective agent is present in the composition in an amount of from about 0.5% to about 35% by weight, based on the weight of the composition.

17. A cosmetic composition comprising:
(a) from about 0.1% to about 3% of an extract of Emblica officinalis;
(b) from about 0.25% to about 15% of glycolic acid;
(c) from about 0.1% to about 5% of 4-(2-hydroxyethyl)piperazine-1-propanesulfonic acid; and
(d) optionally, at least one photoprotective agent.

18. A method of depigmenting skin, comprising applying to the skin, a composition comprising:
(a) an extract of Emblica officinalis;
(b) at least one hydroxyl acid;
(c) at least one N-substituted aminosulfonic acid having the structural formula (I): 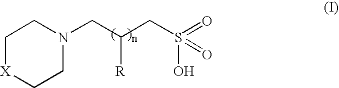
in which R is chosen from a hydrogen atom, —OH or —NH₂; X is chosen from an oxygen atom, a group corresponding to: 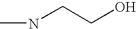
or a group corresponding to: 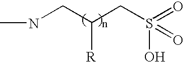
and n is an integer from 0 to 3; and
(d) optionally, at least one photoprotective agent.

19. The method of claim 18, wherein the extract of Emblica officinalis is present in the composition in an amount of from about 0.05% to about 10% by weight, based on the weight of the composition.

20. The method of claim 18, wherein the extract of Emblica officinalis is present in the composition in an amount of from about 0.1% to about 3% by weight, based on the weight of the composition.

21. The method of claim 18, wherein the at least one hydroxyl acid is chosen from α-hydroxy acids, β-hydroxy acids, poly-hydroxy acids and their mixtures.

22. The method of claim 18, wherein the at least one hydroxy acid is glycolic acid.

23. The method of claim 18, wherein the at least one hydroxy acid is present in the composition in an amount of from about 0.1% to about 30% by weight, based on the weight of the composition.

24. The method of claim 18, wherein the at least one hydroxy acid is present in the composition in an amount of from about 0.25% to about 15% by weight, based on the weight of the composition.

25. The method of claim 18, wherein in formula (I) n is equal to 0 or 1.

26. The method of claim 18, wherein the at least one N-substituted aminosulfonic acid is piperazine-1,4-bis(2-ethanesulfonic acid).

27. The method of claim 18, wherein the at least one N-substituted aminosulfonic acid is piperazine-1,4-bis(2-hydroxypropanesulfonic acid).

28. The method of claim 18, wherein the at least one N-substituted aminosulfonic acid is 4-(2-hydroxyethyl)piperazine-1-propanesulfonic acid.

29. The method of claim 18, wherein the at least one N-substituted aminosulfonic acid is present in the composition in an amount of from about 0.1% to about 20% by weight, based on the weight of the composition.

30. The method of claim 18, wherein the at least one N-substituted aminosulfonic acid is present in the composition in an amount of from about 0.1% to about 5% by weight, based on the weight of the composition.

31. The method of claim 18, wherein the photoprotective agent is terephthalylidene dicamphor sulfonic acid.

32. The method of claim 18, wherein the photoprotective agent is drometrizole trisiloxane.

33. The method of claim 18, wherein the photoprotective agent is present in the composition in an amount of from about 0.5% to about 35% by weight, based on the weight of the composition.
